(19) United States
(12) Patent Application Publication
(10) Pub. No.: US 2013/0211038 A1
(43) Pub. Date: Aug. 15, 2013

(54) HIGH TEMPERATURE LACTAM NEUTRALISATION

(71) Applicants: Hendrik Oevering, Beek (NL); Rudolf Philippus Maria Guit, Maastricht (NL); Henricus Anna Christiaan Baur, Herten (NL)

(72) Inventors: Hendrik Oevering, Beek (NL); Rudolf Philippus Maria Guit, Maastricht (NL); Henricus Anna Christiaan Baur, Herten (NL)

(73) Assignee: DSM IP Assets B.V., Heerlen (NL)

(21) Appl. No.: 13/807,995

(22) PCT Filed: Jul. 1, 2011
(86) PCT No.: PCT/EP11/03281

§ 371 Date: Mar. 18, 2013

(30) Foreign Application Priority Data
Jul. 2, 2010 (EP) 10168264.9

(51) Int. Cl. C07D 223/10

(52) U.S. Cl. CPC C07D 223/10
USPC 528/323; 540/533

(57) ABSTRACT

The invention relates to a method for preparing a lactam in a continuous process, comprising forming the lactam and ammonium sulphate by contacting a lactam sulphate contained in an acidic liquid with ammonia, during which forming of lactam heat of reaction is generated, which heat is partially or fully recovered, wherein ammonia is brought into contact with the acidic liquid as part of a liquid aqueous ammonia solution, and wherein the contacting takes place at a temperature of at least 120° C., and wherein the average residence time at a temperature of at least 120° C. is at most 15 minutes, and wherein the ammonium sulphate remains dissolved in a liquid phase during said residence time.

The present invention relates to a method for preparing a lactam, in particular epsilon-caprolactam.

According to known intramolecular rearrangement processes lactams can be obtained from the corresponding cyclic oximes with the use of various acids. This process according to Beckmann (known as a Beckmann rearrangement) is for instance practiced commercially in the preparation of epsilon-caprolactam (hereafter ‘caprolactam’) from cyclohexanone oxime using an acid source, for instance sulphuric acid, in which, ultimately, a reaction mixture containing caprolactam and sulphuric acid, and by-products is obtained.

The lactam synthesised in a Beckmann rearrangement is thus obtained as lactam sulphate in a reaction mixture. In order to separate the lactam from the sulphate, the mixture is usually neutralised with ammonia. The neutralisation is a strongly exothermic reaction. As a result of the neutralisation a layer of “lactam oil” (which is a caprolactam-rich layer, that also might be referred to as Crude Rearranged Oxime) floating on top and of a lower layer mainly consisting of ammonium sulphate in water is usually formed.

After separation of these layers, the lactam and the ammonium sulphate can be recovered.

U.S. Pat. No. 3,907,781 describes a continuous process for the recovery of caprolactam from a synthesis reaction mixture comprising lactam sulphate by concurrently neutralising and crystallising the synthesis reaction mixture, comprising the steps of neutralising the synthesis reaction mixture with ammonia in a circulating volume of ammonium sulphate solution, the neutralization simultaneously forming additional ammonium sulphate crystals in a single stage. The crystal-rich neutralised mixture is passed to a boiling area where the mixture is caused to boil and water vapour is discharged from the mixture, whereby the heat generated is discharged out of the system by evaporation of a portion of the water of the recirculating mixture. The neutralised solution is separated into a supernatant layer lactam-rich aqueous solution and a suspension of ammonium sulphate crystals in ammonium sulphate solution. The lactam-rich layer is recovered and the suspension is separated into an ammonium sulphate crystal fraction and a mother liquor. The separated mother liquor is recycled to the neutralization zone. The process of U.S. Pat. No. 3,907,781 is characterized in avoiding cooling surfaces. Such surfaces are considered disadvantageous because crystals may deposit on it (col 2, lines 15-16).

It is stated in U.S. Pat. No. 3,907,781 that the neutralisation and crystallisation can take place together in a single stage at atmospheric or higher pressure at the relatively high boiling point of the reaction mixture, without any risk of losses through hydrolysis of lactam. Thus, steam (depending on the pressure usually having a temperature above 100° C.) can be produced from the water in the reaction mixture. In the example, the neutralisation took place at 108° C., with an average residence time of 45-60 min.

It is a disadvantage of the process of U.S. Pat. No. 3,907,781 that the steam thus obtained may comprise impurities (e.g. ammonia, sulphur dioxide, and entrained salts) from the reaction mixture from which it is produced, which may limit its applicability. E.g., impurities may form depositions in a steam network via which the steam may be distributed.

Further, the present inventors investigated the effect of maintaining the neutralised mixture at a more elevated temperature than 108° C. on the formation of impurities. They found that at a temperature of, e.g. 130° C. or more, significant impurity formation takes place already within considerably less time, as determined by extinction measurements of a sample of the product stream comprising caprolactam at 290 nm (E₂₉₀). Although the E₂₉₀ measured in those investigations may still be acceptable, it is reasonable to assume that further impurity formation is likely to occur as the residence time is further increased in the methods of the prior art. Accordingly, there is need for a process that allows performing the neutralisation step at higher temperatures than in the prior art process, while still achieving excellent product properties of the lactam and without other problems as mentioned above.

Further, the inventors contemplate that direct crystallisation of ammonium sulphate at a high temperature and in the presence of lactam may adversely affect the quality of the lactam product that is obtained. Also it is contemplated that in a so called open steam generation (as described in U.S. Pat. No. 3,907,781) some ammonium sulphate degradation may occur. It is contemplated in particular that such process cannot be operated with an excess of ammonia. As a result thereof some of the ammonium sulphate may dissociate into ammonia and the extremely corrosive ammonium bisulphate, which will further be decomposed into sulphuric acid and again ammonia. Further, it is in particular contemplated that ammonium sulphate obtained in a prior art process, such as U.S. Pat. No. 3,907,781, may be disadvantageous if the ammonium sulphate crystals obtained are to be used to prepare ammonium sulphate granules.

Thus, it is concluded that the process of U.S. Pat. No. 3,907,781 is disadvantageous when operated at such a high temperature, as it may cause an undesirable or even unacceptable impurity formation in the caprolactam, which impurities may be difficult to remove. It would be desirable though to provide a method that can be carried out at a temperature of 120° C. or more, with reduced risk of substantial impurity formation. This would allow the generation of steam of a higher temperature and thus a higher pressure, which is desirable in particular as a steam supply to a high grade steam network, which can be used to transfer energy from the neutralisation process to a different process.

U.S. Pat. No. 4,021,422 claims to provide an improved process which can be applied at a higher temperature than the process of U.S. Pat. No. 3,907,781 with only slight loss due to increased hydrolysis (column 1, lines 44-52). In order to accomplish this, the process must be carried out without recycling of mother liquor and/or ammonium sulphate crystals. Further, mixing is said to be improved owing to boiling phenomena due to heat of neutralisation in the reaction mixture. This publication also teaches away from using a heat exchanger, because it is considered that crystals may deposit. Further, it is stated that temperature control would be better than when using a heat exchanger in which heat is removed by cooling water.

In Example I of U.S. Pat. No. 4,021,422, the neutralisation was carried out in two steps. First the rearrangement mixture was neutralised at 150° C. for about 20 min. Next, the phase comprising ammonium sulphate was introduced into a second neutraliser which was operated at atmospheric pressure and 180° C. Steam of high-temperature and super-atmospheric pressure is only generated in the first neutraliser. Thus, it is apparent that a substantial amount of the generated neutralisation heat is not made available for steam production.

In addition, from the investigations by the present inventors mentioned above, it was concluded that also a neutralisation at 150° C. for about 20 min. would also likely result in substantial impurity formation.

Further, the method of U.S. Pat. No. 4,021,422 would not allow recycle of mother liquor, which may be disadvantageous for the product yield, unless additional equipment is used to treat the mother liquor.

Furthermore, as the steam is generated directly from the reaction mixtures, the same considerations apply regarding the presence of impurities as for U.S. Pat. No. 3,907,781.

It is an object of the present invention to provide a novel method for preparing lactam which can serve as an alternative to known methods, in particular a novel method that overcomes one or more of the disadvantages of the prior art cited herein above.

It has now been found that a lactam can adequately be produced by neutralising a liquid containing a lactam sulphate under specific neutralisation conditions.

Accordingly, the present invention relates to a method for preparing a lactam in a continuous process, comprising forming the lactam and ammonium sulphate by contacting a lactam sulphate contained in an acidic liquid with ammonia, during which forming of lactam heat of reaction is generated, which heat is partially or fully recovered, wherein ammonia is brought into contact with the acidic liquid as part of a liquid aqueous ammonia solution, and wherein the contacting takes place at a temperature of at least 120° C., and wherein the average residence time at a temperature of at least 120° C. is at most 15 minutes, and wherein the ammonium sulphate remains dissolved in a liquid phase during said residence time.

The inventors surprisingly found that it is possible to accomplish the preparation of the lactam from lactam sulphate at an elevated temperature requiring only a short residence time at elevated temperature (a temperature of 120° C. and more), whilst avoiding undesired crystallisation of ammonium sulphate in a space wherein the lactam sulphate and the liquid aqueous ammonia are brought into contact with each other. Typically, the exposure to said elevated temperature during said residence time in accordance with the invention takes place without letting the acidic liquid respectively the process stream formed by bringing the liquid aqueous ammonia and acidic liquid into contact, boil. This is desired in view of avoiding undesired crystallisation and/or blocking/fouling of the equipment that is used.

As indicated above, the ammonium sulphate is obtained dissolved in a liquid phase rather than as precipitated crystals. The lactam is generally also obtained as part of a liquid phase, which liquid phase may be the same as or different from the liquid phase wherein the ammonium sulphate is present. The lactam and the ammonium sulphate generally leave the space wherein it has been formed as part of the same or a different liquid effluent stream. Under the process conditions of the process according to the invention the stream remains liquid.

The method of the invention allows recovery of the heat of reaction such that this heat may be used fully or partially for a useful purpose. The heat may be used directly to heat a process stream, which may for instance be a process stream of another method for preparing a chemical compound or a process stream of a method for further processing a product stream (e.g. a separation method such as distillation, or crystallisation) or the heat may be partially or fully recovered in a method comprising transferring said heat to a heat exchange medium, which may be a liquid such as oil or water, or a gas, such as steam.

A method according to the invention is in particular suitable for generating steam, more in particular super-atmospheric steam. The super-atmospheric steam that is generated preferably is high-pressure steam (having a pressure of at least 2 atm., in particular having a pressure of 2-10 atm.) of a high temperature, such as steam having a temperature of at least 120° C., at least 130° C., at least 140° C., at least 150° C. or even at least 160° C.

The invention is further advantageous in that it does not require the boiling of any liquid phase that is formed in the method of preparing the lactam in order to generate heated steam. Boiling of a liquid phase comprising the lactam or lactam sulphate at a high temperature for a prolonged time is undesired because it may cause side-product formation or precipitation of product or side product. Also, it is advantageous because the heating medium, e.g. water to which the heat of reaction may be transferred does not need to originate from the reaction in accordance with the invention, and thus may be clean. If desired, in a subsequent step, ammonium sulphate crystallisation may be carried out in a step wherein solvent (water) is evaporated. This may involve boiling of the phase comprising ammonium sulphate, which can be performed at a relatively low temperature (<100° C., under reduced pressure), or at a temperature of above 100° C., even in the range of from 110 to 116° C. At higher pressure even higher temperatures can be reached.

Thus, a method according to the invention may in particular be used for generating or (re-)heating steam or another heat exchange medium of a heating network, such as a steam network or another heat exchange medium network that is used for heating purposes, remote from the place where the heat is generated.

The term “or” as used herein means “and/or” unless specified otherwise.

The term “a” or “an” as used herein means “at least one” unless specified otherwise.

When referring to a noun (e.g. a compound, an additive etc.) in singular, the plural is meant to be included, unless specified otherwise.

As used herein the “residence time” can be calculated as the volume of the space wherein the contacting at a temperature of at least 120° C. takes place (in litre) divided by the total feed rate of liquids into the space (generally the sum of litres/min. of acidic liquid and litres/min. of ammonia containing liquid).

Methods to provide lactam sulphate for use in a method of the invention are generally known in the art, see e.g. “Ullmann's encyclopedia of Industrial Chemistry”, for instance in the fifth edition (1986), Volume A5, pages 38-39. It is noted that the same information is still mentioned in the 2005 edition of Ullmann (7th Edition), which is electronically available for subscribers, in particular in the part “Caprolactam””. The lactam concentration in the acidic liquid is not critical, but in practice is usually in the range of 20 to 70 wt. %, in particular 40 to 60 wt. %, more in particular about 50 wt. %. As the skilled person knows, the acidic liquid usually also comprises sulphuric acid, as the formation of lactam sulphate usually is carried out in excess of sulphuric acid. The molar ratio of lactam, in particular caprolactam, to H₂SO₄ (including dissociated forms thereof)+SO₃ in the acidic liquid usually is in the range of 1.1 and 2.0.

The lactam that is prepared may in particular be selected from the group of lactams having 6-12 carbon atoms, more in particular from the group of caprolactam, octalactam, nonalactam, decalactam, undecalactam and laurolactam. A preferred lactam is caprolactam.

The total amount of liquid aqueous ammonia contacted with the acidic liquid in the process of forming the lactam is usually at least a stoichiometric amount, i.e. at least 2 times the number of moles of sulphate equivalents (sulphate in lactam sulphate, sulphuric acid and ionised forms thereof). A more than stoichiometric amount may be advantageous for a high caprolactam recovery. In practice, the acidic liquid comprising lactam sulphate originating from a Beckmann rearrangement, comprises an excess of sulphate/sulphuric acid and may comprise sulphite/sulphurous acid. The amount of ammonia added is preferably sufficient to also react with these compounds in the liquid.

The liquid aqueous ammonia is brought into contact with the acidic liquid in a solution form. Adding ammonia in aqueous solution instead of in gaseous form is advantageous because addition of ammonia in gaseous form leads to undesired crystallisation of ammonium sulphate.

The ammonia concentration in the liquid can in principle be chosen freely, e.g. in the range of 5-50 wt. %. The total amount of fed liquid aqueous ammonia solution is preferably regulated, based on the apparent pH (pH as measured by a pH meter) in the (aqueous) ammonium-rich liquid phase which is formed in a method of the invention after phase separation into a lactam-rich phase and an ammonium sulphate-rich phase and after neutralisation of the ammonium sulphate-rich phase. This pH preferably is maintained in the range of 2-6, in particular in the range of 4-5. As will be understood by the skilled person, an increase in ammonia feed is suitable to increase pH and a decrease in ammonia feed is suitable to decrease pH. In this pH range, essentially full conversion of sulphuric acid and sulphur trioxide into ammonium sulphate is achieved, whilst avoiding a significant excess of unreacted ammonia.

The amount of water that is fed (as part of the liquid comprising ammonia and as part of the acidic liquid) is chosen such that the ammonium sulphate concentration during the residence time is below its crystallisation concentration (crystallisation point) under the reaction conditions, preferably at least about 2% below its crystallisation point; thus, in a method wherein the crystallisation point is 44 wt. %, the concentration preferably is about 43 wt. % or less. On the other hand, it is preferred to maintain the ammonium sulphate concentration relatively high, in view of energy efficiency, and processing time, when the ammonium sulphate is to be crystallised in a later step. Accordingly, the ammonium sulphate concentration preferably is at least 75% of the crystallisation point, in particular at least about 85% of the crystallisation point, for instance about 90% of the crystallisation point; thus, for a method with a crystallisation point of 44 wt. %, the concentration preferably is at least 33 wt. %, in particular at least about 37 wt. %, for instance about 40 wt. %.

The liquid aqueous ammonia and the acidic liquid comprising lactam sulphate may continuously be brought into contact at a single feed-introduction-point, wherein both the acidic liquid feed and the ammonia feed are integrally brought into contact with each other or they may be brought into contact with each other portion wise. This is generally accomplished by dividing at least one of said feeds in two or more partial feeds and introducing the partial feeds at multiple feed-introduction-points into the space wherein the acidic liquid and liquid aqueous ammonia are contacted, wherein each subsequent feed-introduction-point is situated down-stream of a previous feed-introduction point. This principle may also be referred to as multi-point injection. Another form of multi-point injection, which may be combined with the aforementioned principle, is perpendicular injection over, for instance, a ring. It has been found that portion wise addition of liquid aqueous ammonia or acidic liquid is advantageous with respect to a low tendency for side-production formation determined by measuring E₂₉₀.

In particular, this may be accomplished by feeding part of the acidic liquid to the liquid aqueous ammonia feed at a first feed-introduction point, thereby forming a first reaction stream and thereafter feeding a further part of the acidic liquid to the reaction stream, in a second feed-introduction point, downstream of the first feed-introduction point or by feeding part of the ammonia to the acidic liquid feed at a first feed-introduction point, thereby forming a first reaction stream and thereafter feeding a further part of the ammonia to the first reaction stream, in a second feed-introduction point, downstream of the first feed-introduction point, thereby forming a second reaction stream.

In a first embodiment a device with combined mixer/reactor properties is followed by a cooler. This embodiment may be called a single step process. Alternatively, in another form of such single step process, a device with combined mixer/reactor/cooler properties is used.

In further specific embodiments, two or more devices (with combined mixer/reactor optionally combined with cooler properties) are used in series. Embodiments comprising two of such devices in series (each of which either is followed by a cooler, or already incorporates cooling properties) may be called a two-step process. Similarly, embodiments comprising three of such devices in series (each of which either is followed by a cooler, or already incorporates cooling properties) may be called a two-step process.

Accordingly, all such embodiments part of the aqueous ammonia is mixed with the acidic liquid comprising lactam sulphate in the first device (or, in case of the single-step process, the sole device used). If a two- or three-step process is performed, then again part of the aqueous ammonia is mixed with the acidic liquid comprising lactam sulphate in the second, or third device. The resultant mixture after the first step of a two-step process, respectively after the second step of a three-step process (comprising lactam, dissolved ammonium sulphate and lactam sulphate that has not been converted if conversion is incomplete) is fed into the next device, where further aqueous ammonia is added, or is fed preferably after cooling the product mixture in an after-cooler to a temperature level of below 120° C., preferably of at most 100° C.—into a phase separator wherein the formed lactam-rich phase and aqueous ammonium sulphate-rich phase are separated from each other. It is to be noticed that the ammonia feed added to the third device, if present, and/or to the second device, if present, does not need to be an aqueous solution as added into the first device. The ammonia in such steps may be of a higher concentration if used as aqueous ammonia or even may be gaseous.

As indicated above, according to the present invention the contacting takes place at a temperature of at least 120° C. In particular in case at least part of the heat of reaction is used for heating water or another heat exchange medium while producing steam, said temperature may advantageously be higher, thus allowing to heat the heat exchange medium to a more elevated temperature and/or to heat the heat exchange medium faster. Thus, said temperature preferably is at least 130° C., at least 140° C., at least 150° C., or at least 160° C. The temperature, however, should be below the boiling temperature of the acidic liquid (under the existing conditions) and of the lactam formed. As will be understood by the skilled person, the boiling temperature can be increased by increasing the pressure under which the process of forming the lactam by neutralisation of a lactam sulphate stream is carried out.

In general, the heat of reaction will cause the liquid wherein lactam and ammonium sulphate have formed to increase in temperature. The highest temperature that is reached at any point during the residence time is called the process peak temperature. In general, the process peak temperature is at least 130° C., in particular at least 140° C., more in particular at least 150° C., or at least 160° C. A higher process peak temperature allows heating of a heat exchange medium or another process stream to a higher temperature and/or higher pressure. Usually, the process peak temperature is 325° C. or less. For a good product quality or yield (less side-product formation) and/or more flexibility with respect to process conditions under which undesired crystallisation is relatively easily avoided, the process peak temperature preferably is 250° C. or less, in particular 200° C. or less, more in particular 190° C. or less, or 180° C. or less. In particular for the above reasons, in specifically preferred embodiments the process peak temperature is in the range of 140-250° C., 150-200° C., 160-190° C. or 160-180° C.

It should be noted that it is not essential that the acidic liquid and the liquid aqueous ammonia as they become available for use in the present process of the invention, already are at a temperature of 120° C. or more initially. These feed streams namely may be pre-heated to a temperature of 120° C. or more before they are actually brought into contact with each other at such temperature of 120° C. or more. E.g. if the acidic liquid is obtained in a Beckmann rearrangement at a temperature of about 70° C., optionally heated with heat generated in the Beckmann rearrangement (or in another process where heat is generated and recovered, for instance the process of the present invention itself) and directly led into the method of the present invention, the temperature may increased by such pre-heating to over 120° C., for instance about 130° C. or higher. This may be advantageous in view of energy-efficiency.

Once they have been brought into contact, the lactam sulphate and the ammonia will react, causing considerable heat of reaction which will cause the temperature to rise to a temperature over 120° C., or even above 140° C. or more. By appropriate heat-exchange the residence time at a temperature of at least 120° C. is at most 15 min. and thus, after maximally 15 min. from reaching a temperature of at least 120° C., the temperature of the stream comprising lactam and ammonium sulphate that is formed is reduced to a value below 120° C. In a specifically preferred method the residence time at a temperature of at least 120° C. is 10 min. or less, 5 min. or less, 2 min. or less, 1 min. or less, 30 sec. or less, or 20 sec. or less. It is contemplated that a residence time of 1 sec. or less is sufficient for obtaining the lactam. Thus, minimum residence times are in general determined by the equipment used, as will be understood by the skilled person. Accordingly, for practical reasons, the residence time usually is 1 sec. or more, in particular at least 5 sec., at least 10 sec., at least 30 sec., at least 1 min. or at least 2 min.

A relatively low residence time is in particular considered advantageous in that the formation of undesired side-products may be reduced. Without being bound by any theory, it is contemplated that the higher the temperature, the lower a specifically preferred residence time may be. As a rule of thumb, it is contemplated that specifically preferred residence times, as mentioned above, are reduced by about a factor 2 per 10° C. increase in the temperature, with the proviso that the minimum residence time usually is about 1 sec. or more. Depending on the specific conditions and desired product quality, the skilled person will be able to determine particularly suitable conditions based on the information disclosed herein, common general knowledge and optionally some routine testing. For instance, if it is found that under specific conditions at a specific temperature and residence time too many side-products are formed in order to meet a particular specification with respect to product quality (as may be determined by UV-extinction measurements at 290 nm (E₂₉₀) in a manner known per se, the skilled person may reduce the residence time, the temperature, contact less ammonia with the lactam sulphate. In view of this, it has for instance been considered advantageous to have a residence time at at least 140° C. of at most 10 min., in particular of at most 70 sec.; to have a residence time at at least 160° C. of at most 140 sec., in particular of at most 70 sec.; or to have a residence time at at least 180° C. of at most 35 sec., in particular of at most 20 sec.

In accordance with the invention, the ammonium sulphate that is formed remains dissolved in the liquid phase for at least the residence time. As used herein ‘dissolved’ means that essentially no ammonium sulphate precipitates are present. Preferably, during the contacting at the temperature of at least 120° C. no detectible crystallisation of ammonium sulphate of ammonium sulphate takes place. This can be accomplished by taking care that the ammonium sulphate concentration remains below saturation concentration under the given conditions. The skilled person will be able to take care of this, based on common general knowledge and the information disclosed herein without undue burden.

The contacting of lactam sulphate and liquid aqueous ammonia may be carried out in a mixing unit for mixing fluids known in the art per se. For example use may be made of one or more static mixers or in-line mixers. Suitable mixing units are in particular:
Static mixer reactors (Re-engineering the chemical processing plant: process intensification, A. Stankiewicz, J. Moulijn, 2004, Marcel Dekker Inc.);
Micro mixers, micro reactors (Transport phenomena in micro process engineering, N. Kockmann, Springer, 2008, chapter 5, “Diffusion, mixing, and mass transfer equipment”);
Helical tube reactors (WO 2009/51322A1);
Rotor-stator reactors (Boume J. R. and Studer M., 1992, Fast reactions in rotor-stator mixers of different size. Chemical Engineering and Processing 31:285-296.);
Spinning disk reactors (Re-engineering the chemical processing plant: process intensification, A. Stankiewicz, J. Moulijn, 2004, Marcel Dekker Inc.); HEX reactor (Edge, A M, Pearce, I and Phillips C H “Compact heat exchangers as chemical reactors for process intensification (PI)”, 2nd International Conference on Process Intensification for the Chemical Industry, Antwerp, 1997);
Sulzer SMR™ mixer, for mixing and heat exchange in a single apparatus.

Micro-mixers are in particular useful for providing a method with a particularly short residence time, if desired. Also, such mixers are particularly useful for recovering heat whilst the acidic liquid and ammonium sulphate are brought into contact. For such method an integrated micro-device can be used that comprises a mixer, a reactor and a heat-exchanger, optionally with an after-cooler for the product stream comprising lactam and ammonium sulphate downstream of the heat-exchanger. Furthermore, micro-mixers are in particular useful for a method with a relatively high process peak temperature, whilst maintaining a good product quality and yield.

Advantageously, a mixing unit used for bringing the acidic liquid and the liquid aqueous ammonia into contact has a mixing time of at most 50% of the residence time. The minimum mixing time is not critical and can be any value larger than 0 sec., e.g. the mixing time may be at least 0.01% of the residence time, at least 0.1% of the residence time or at least 1% of the residence time. The term ‘mixing time’ is as defined in “Micro mixers, micro reactors (Transport phenomena in micro process engineering)”, N. Kockmann, Springer, 2008, chapter 5, “Diffusion, mixing, and mass transfer equipment”.

Advantageously, the reaction takes place in a device comprising a reactor unit made of a material with a high heat-conductivity an a high corrosion resistivity. Preferred examples of such materials are SiC, AlN 4,4, AlN 3,3, Hastelloy steel, and other materials having a similar or better heat conductivity and/or similar or better corrosion resistivity. Materials having good corrosion resistance are preferred.

By increasing the number of mixers, the process peak temperature is usually reduced. As a rule of thumb the number of mixers and intermediate coolers (N) is proportional to the adiabatic temperature rise of the process liquid (i.e. the mixture formed from the liquid aqueous ammonia and acidic liquid that are contacted with each other).

The contacting (mixing) of acidic liquid comprising lactam sulphate and the liquid aqueous ammonia and the heat exchange can be done simultaneously (using a system wherein the contacting space is provided with a heat exchanger) or sequentially (with the unit providing the contacting space and the heat exchanger being positioned in series, the heat exchanger being down stream).

In principle, the transfer of reaction heat can be accomplished in any way.

Preferably the heat, or at least a substantial part thereof (preferably >50%, in particular >80%), is transferred via a heat exchanger, which can be integrated with the space wherein lactam is formed. For instance, this space can be defined at least partially by one or more outer walls of the heat exchanger or the formation may be carried out in a mixing unit of which one or more walls are in thermally conductive contact with the heat exchanger wherein the heat is transferred to a heat exchange medium. Thus, heat is transferred as the lactam is being formed. Such method, especially when combined with introducing the partial feeds at multiple-feed injection points and using one mixer/reactor followed by cooler device, or some of these devices in series, may in particular be advantageous to ensure that the process peak temperature is relatively low compared to the temperature reached in a configuration of a single vessel with separate mixing and cooling in a method wherein the heat exchanger is down stream of the space wherein lactam is formed, and may in particular be preferred in an embodiment wherein the contacting is carried out at a relatively high temperature and/or under conditions at which the rate at which heat of reaction is formed is relatively high. Also such embodiment may be advantageous to achieve relatively short residence times.

Alternatively, or in addition, a heat exchanger may be used downstream of the space wherein the lactam sulphate and ammonia have been brought into contact with each other. In such embodiment, the (liquid) effluent stream or streams comprising the lactam respectively ammonium sulphate leaving said space are introduced into the heat exchanger.

The heat exchanger makes it possible to transfer the heat of reaction to a heat exchange medium without having to physically bring the lactam and/or ammonium sulphate in to contact with the heat exchange medium, thus avoiding contamination of the heat exchange medium with lactam, ammonium sulphate or any side-product in the effluent stream. In principle any heat exchange medium can be used, such as steam, liquid water or an organic liquid, for instance an oil, such as a silicon oil, or can be another process flow with which heat is exchanged. A method according to the invention is particularly suitable to (re-)heat steam, more in particular to (re-)heat steam of a high energy heat steam network, or to generate steam, in particular high energy heat steam from liquid water. As will be understood by the skilled person, the temperature and pressure of the steam obtained will depend on factors such as initial temperature and pressure of the steam, the amount of steam, and the amount of heat generated. The steam obtained in accordance with the invention may in particular have a temperature in the range of 130-200° C., with the proviso that the temperature will usually be below the highest temperature the reaction mixture containing lactam and ammonium sulphate reaches (unless the obtained steam is subjected to a compression step). The invention is in particular suitable to provide steam having a pressure of 2-10 bar.

In a specifically preferred embodiment, steam is generated from liquid water. An advantage of this embodiment over re-heating steam is that a smaller heat-exchange surface is needed than for a vapour-liquid heat exchanger (needed for reheating steam). Namely, for generating steam from water, a liquid-liquid heat exchanger can be used wherein the liquid that is heated is subjected to boiling.

Typically the temperature of the heat exchange medium will be lower than the temperature of the phase or phases from which the heat of reaction is transferred (the contents of the space wherein the contacting takes place, or the effluent(s) from said space, comprising lactam and ammonium sulphate). Typical temperature differences that are used depend on the equipment used, as will be understood by the skilled person. Usually the temperature of the heat exchange medium is at least 0.01° C. lower, in particular at least 0.1° C. lower, more in particular at least 0.5° C. lower. In principle, the temperature difference can be very large, e.g. 30° C. or more, but it is contemplated that for efficient use, the temperature of the heat exchange medium advantageously is up to 20° C. lower than the temperature preferably up to 10° C. lower, in particular up to 5° C. lower, more in particular up to 2° C. lower. Thus, high-temperature-high pressure-steam can be generated, having a temperature of e.g. at least 120° C., at least 150° C. or at least 180° C. It should be noted that although the temperature of the steam will generally not exceed the temperature in the space wherein lactam sulphate and ammonia are contacted, as a direct result of the heat transfer, the temperature of the generated steam may be increased above that temperature by compressing the steam.

In a specific embodiment, heat is transferred from the product stream comprising lactam and ammonium sulphate, and this product stream is thereafter subjected to (further) cooling, preferably to a temperature below 100° C., in particular to a temperature of 80° C. or less. The product stream may be cooled to ambient temperature (e.g. about 25° C.) or a higher temperature. The cooling step is in particular advantageous in order to suppress any undesired side-reactions in the product stream, to facilitate phase separation into a lactam-rich phase and an ammonium sulphate-rich phase, or in as far as such phase separation has already occurred to improve ammonium sulphate yield in the ammonium sulphate-rich phase or lactam yield in the lactam-rich phase.

The (further) cooling of said product stream is advantageously performed prior to subjecting the product stream to a separation step, wherein a lactam-rich phase and an ammonium sulphate-rich phase are separated from each other (see also below). It is also possible to first subject the product stream to a separation step wherein an ammonium sulphate-rich phase and a lactam-rich phase are separated from each other, and thereafter subjecting one or both of said phases to cooling.

After formation of the lactam and the ammonium sulphate, a lactam-rich phase and an ammonium sulphate-rich phase are formed. The phase separation may occur essentially instantly as the lactam and ammonium sulphate are being formed or subsequently, depending on the reaction conditions (temperature, concentration of products, pH), as will be understood by the skilled person. For instance, phase separation may occur as the lactam and ammonium sulphate are being formed at a sufficiently high concentration of lactam and ammonium sulphate. Subsequent phase-separation is usually accomplished by reducing the temperature to a temperature at which process streams are chemically stable and at which phase separation occurs. Suitable conditions are commonly known in the art. For caprolactam, cooling to a temperature of 80° C. or less is in general suitable. Preferably, for caprolactam, a caprolactam-rich phase is formed containing between 60% caprolactam and the saturation concentration of caprolactam in water. Preferably, an ammonium sulphate-rich phase is formed containing between 30 wt. % and saturation concentration of ammonium sulphate in water.

The separated phases may be isolated from each other in a manner known per se. Any of the phase separation, isolation, and further processing of the isolated phases may carried out in the same continuous process as the formation of the lactam.

After isolation of the lactam-rich phase, the lactam can be recovered from the lactam-rich phase in a manner known per se, e.g. by liquid extraction with benzene, toluene, or another extraction medium. After recovery the lactam may further be purified. Suitable purification techniques, such as those comprising distillation and/or crystallisation are also commonly known in the art.

It is observed that a method according to the invention allows essentially full conversion of lactam sulphate to lactam, within said residence time, provided that at least a stoichiometric amount of ammonia is contacted with the lactam sulphate, in particularly preferred embodiments by carrying out the process of the invention as a two-step or three-step process. It is also possible to carry out the method under conditions wherein after the residence time the conversion is not complete. Usually, the conversion is 90-100%, in particular 95-100%. In a specific embodiment, the method is carried out to have a conversion lactam sulphate to lactam of 99% or less, or 98% or less. A method wherein conversion during the residence time is incomplete is considered advantageous in order to facilitate process control stability.

In case of incomplete conversion during the residence time, remainder of the lactam sulphate may be reacted with ammonia to provide lactam and ammonium sulphate in a subsequent step. This reaction may be carried using only the lactam-rich phase after phase separation of the product stream comprising lactam and ammonium sulphate into a lactam-rich phase and an ammonium sulphate-rich phase, and after the lactam-rich phase has been separated from the ammonium sulphate-rich phase, if desired. This may be done in a manner known per se, for example in a continuously stirred tank reactor or in a recycle cooler. It is preferred, however, that both phases (lactam-rich phase and ammonium sulphate-rich phase) are combined for the neutralisation. An after-treatment wherein remaining lactam sulphate is converted into lactam is generally carried out at a temperature below 120° C.

The lactam obtained in according to the invention may in particular be used in the preparation of a polymer, preferably a polyamide. Suitable methods for preparing a polymer using the lactam, in particular caprolactam, as a monomer are generally known in the art.

If desired, ammonium sulphate may be recovered from the liquid phase. Typically, recovery comprises crystallising the ammonium sulphate after isolating the ammonium sulphate-rich phase from the lactam-rich phase. The skilled person will know how to cause crystallisation, based on common general knowledge. Crystallisation is usually accomplished by a treatment whereby the ammonium sulphate concentration exceeds the saturation concentration. This is usually accomplished by evaporating water from the liquid phase.

In particular, in a method according to the invention, an ammonium sulphate-rich aqueous phase and a lactam-rich phase are formed, which phases are separated from each other, after which the first is subjected to a crystallisation step, whereby ammonium sulphate crystals and a mother liquor are formed, and wherein the crystals are isolated from the mother liquor. The crystallisation step usually takes place at a temperature below 200° C., in particular at a temperature in the range of 30-160° C., more in particular at a temperature in the range of 40-120° C.

The ammonium sulphate may further be processed in a manner known per se, and be used, e.g. as a fertiliser.

The invention is now illustrated by the following examples.

### EXAMPLE 1

A Beckmann rearrangement mixture with a molar ratio of 1.6 mol/mol (mol H₂SO₄+SO₃/mol caprolactam) and an aqueous ammonia solution (10 wt % NH₃) was fed to an in-line stainless steel T-mixer. Both feeds, each available at 70° C., were heated to 130° C. while being fed into the T-mixer. The T-mixer and subsequent mixing and reaction zone were placed in an oil-bath that was controlled at a fixed reaction temperature. The mixing and reaction zone consisted of a 75 cm stainless steel tube having an internal diameter of 1 mm. The outlet of the mixing and reaction zone was connected to a cooling zone consisting of a 50 cm stainless steel tube having an internal diameter of 1 mm. This zone was placed in a cooling bath. This zone was always controlled to be at a temperature of about 20° C. at the outlet of the cooling zone. In-line thermocouples were used to measure and control the local process temperatures. The mixing, reaction and cooling zones were held under pressure to avoid gas formation and keep the reactor contents in liquid form under all circumstances. After the cooling zone, the product was depressurized and collected in a vessel at ambient temperature. Here the product was separated in two liquid phases, the bottom phase being an aqueous solution rich in ammonium sulphate (appr. 30-40 w %). The top phase was a caprolactam-rich product oil. The feed-rate of the aqueous NH₃ feed stream to the mixer was adjusted to obtain a pH of approximately 4-5 in the aqueous product phase rich in ammonium sulphate.

While the mixing and reaction zone were held at 130° C. at the outer surface of the T-mixer, feed-rates were adjusted for an overall residence time in the mixing and reaction zone varying from 2 to 20 seconds.

TABLE 1
Results of experiments described under Example 1

| Residence time (in seconds) | Reaction temperature | E₂₉₀ |
|---|---|---|
| 20.2 | 130 | 0.785 |
| 10.1 | 130 | 0.781 |
| 2.4 | 130 | 0.756 |

### EXAMPLE 2

A different Beckmann rearrangement mixture with a molar ratio of 1.35 mol/mol (mol H₂SO₄+SO₃/mol caprolactam) and an aqueous ammonia solution (10 wt % NH₃) was fed to a pressurised continuous stirred reactor applying a stirring speed of 1000 rpm. The reaction was carried out at a constant temperature of 160° C. The outlet of the reactor was cooled in two stages to room temperature. In-line thermo couples were used to measure and control the local process temperatures. The mixing, reaction and cooling zones were held under pressure to avoid gas formation and keep the reactor contents in liquid form under all circumstances. After the cooling zone, the product was depressurized and collected in a vessel at ambient temperature. Here the product was separated into two liquid phases, the bottom phase being an aqueous solution rich in ammonium sulphate (appr. 30-40 w %). The top phase was a caprolactam-rich product oil. The feed-rate of the aqueous NH₃ feed stream to the mixer was adjusted to obtain a pH of approximately 4-5 in the aqueous product phase rich in ammonium sulphate.

Feed-rates of both feeds were adjusted to achieve overall residence times in the reactor varying from 4 to 30 minutes.

TABLE 2
Results of experiments described under Example 2

| Residence time (in minutes) | Reaction temperature | E₂₉₀ |
|---|---|---|
| 4 | 160 | 2.159 |
| 8 | 160 | 2.234 |
| 20 | 160 | 2.390 |
| 30 | 160 | 2.590 |

### EXAMPLE 3

### Comparative Example to Example 1

In this example, the reaction zone was extended to make long residence times at high temperature possible. With this extended reaction zone the experiment of Example 1 was repeated at 130° C. using the same starting material as in Example 1. Liquid residence times in the mixing and reaction zone were varied from 30 minutes to 240 minutes. The E₂₉₀ of the caprolactam product layer obtained in these experiments increased from 1.06 at 30 min. residence time to 1.27 at 240 min. residence time. These experiments show that E₂₉₀ is significantly influenced by prolonged residence times.

### EXAMPLE 4

### Comparative Example to Example 2

In this example the same method and starting material (Beckmann rearrangement mixture) was used as in the experiments described in Example 2. In this case the reaction temperature was kept at 50° C. while other conditions were the same.

TABLE 3
Results of experiments described under Example 4

| Residence time (in minutes) | Reaction temperature | E₂₉₀ |
|---|---|---|
| 8 | 50 | 1.994 |

Examples 3 and 4 show that prolonged residence times in the mixing and reaction zone have a negative effect on E₂₉₀, but by strong reduction of the residence time it is possible to increase reaction temperature without an unacceptable negative effect on the E₂₉₀.

1. Method for preparing a lactam in a continuous process, comprising forming the lactam and ammonium sulphate by contacting a lactam sulphate contained in an acidic liquid with ammonia, during which forming of lactam heat of reaction is generated, which heat is partially or fully recovered, wherein ammonia is brought into contact with the acidic liquid as part of a liquid aqueous ammonia solution, wherein the contacting takes place at a temperature of at least 120° C., and wherein the average residence time at a temperature of at least 120° C. is at most 15 minutes, and wherein the ammonium sulphate remains dissolved in a liquid phase during said residence time.

2. Method according to claim 1, wherein the heat is partially or fully used for steam generation, preferably steam having a super-atmospheric pressure.

3. Method according to claim 2, wherein the heat is recovered by transferring the heat to a water stream via a heat exchanger, which water stream is converted into a steam stream.

4. Method according to claim 2, wherein heat is transferred from product stream comprising lactam and ammonium sulphate, and the product stream is thereafter subjected to (further) cooling to a temperature below 120° C., preferably below 100° C., in particular to a temperature of 80° C. or less.

5. Method according to claim 1, wherein the average residence time of the formed mixture at a temperature of at least 120° C. is at most 10 min., in particular at most 5 min., more in particular at most 2 min.

6. Method according to claim 1, wherein the contacting of the acidic liquid and the liquid aqueous ammonia is carried out via multi-point injection of acidic liquid or liquid aqueous ammonia.

7. Method according to claim 1, wherein an ammonium sulphate-rich phase and a lactam-rich phase are formed, which phases are separated from each other, after which the first phase is subjected to a crystallisation step, whereby ammonium sulphate crystals and a mother liquor are formed, and wherein the crystals are isolated from the mother liquor.

8. Method according to claim 7, wherein the crystallisation step takes place at a temperature below 200° C., in particular in the range of 30-160° C., more in particular in the range of 40-120° C.

9. Method according to claim 1, wherein the conversion of lactam sulphate into lactam after the contacting at a temperature of at least 120° C. is incomplete, in particular in the range of 90-99%, and wherein remaining lactam sulphate is brought into contact with ammonia, thereby forming lactam and ammonium sulphate, at a temperature below 120° C.

10. Method according to claim 1, wherein the lactam is recovered and subjected to one or more further purification steps.

11. Method according to claim 1, wherein the lactam is epsilon-caprolactam.

12. Use of a lactam obtained in a method according to claim 1 for the preparation of a polymer.
